(19) United States
(12) Patent Application Publication
(10) Pub. No.: US 2026/0086261 A1
(43) Pub. Date: Mar. 26, 2026

(54) OIL-WATER IDENTIFICATION METHOD BASED ON CONDUCTIVITY DISPERSION CHARACTERISTICS OF UNDISTURBED FORMATION

(71) Applicant: Yangtze University, Jingzhou (CN)

(72) Inventors: Wei XU, Jingzhou (CN); Ming CAI, Jingzhou (CN); Xiaolu ZHU, Jingzhou (CN); Hao LI, Jingzhou (CN); Zehua YAN, Jingzhou (CN); Yaping ZHENG, Jingzhou (CN); Yongsong ZHENG, Jingzhou (CN); Weiping WU, Jingzhou (CN)

(73) Assignee: Yangtze University, Jingzhou (CN)

(21) Appl. No.: 19/209,846

(22) Filed: May 16, 2025

(30) Foreign Application Priority Data
Sep. 25, 2024 (CN) 202411338544.9

(51) Int. Cl. G01V 3/38; G01N 33/28; G01V 3/28

(52) U.S. Cl. CPC G01V 3/38; G01N 33/2823; G01N 33/2847; G01V 3/28

(57) ABSTRACT

An oil-water identification method based on RPFE characteristics of undisturbed formation can identify oil and water layers, and the oil-water identification method is based on the dispersion of the original signal of array induction logging, mapping a spectrum image of the array induction logging sub-array 7, and forming a two-dimensional oil-water identification chart by using the dispersion and the product of the resistivity after borehole correction and the formation porosity, and the test of actual logging data shows that this method can accurately and effectively divide the oil and water layers, and improve the efficiency of the oil-water evaluation in reservoir.

### CROSS REFERENCE TO THE RELATED APPLICATIONS

This application is based upon and claims priority to Chinese Patent Application No. 202411338544.9, filed on Sep. 25, 2024, the entire contents of which are incorporated herein by reference.

### TECHNICAL FIELD

The present invention relates to the field of hydrocarbon reservoir evaluation technology, particularly to an oil-water identification method based on conductivity dispersion characteristics of undisturbed formation.

### BACKGROUND

Reservoir oil-water identification is a key field of petroleum exploration and development, and array induction logging is a commonly used logging method for oil-based mud or mud-free dry wells. Array induction logging technology appeared in the early 1990s, compared to conventional dual induction logging, array induction logging has more measurement frequencies and coil system sub-arrays, and the measured formation resistivity information is more abundant, it can be more effective to divide the thin interbed interface, determine the formation radial invasion characteristics, calculate formation oil saturation and identify the gas layer.

However, the conventional oil-water evaluation in reservoir mainly depends on the resistivity logging curve, this method has some limitations (such as high resistivity water layer and low resistivity oil layer appearing due to the influence of formation porosity and other factors), thus posing a certain difficulty in the oil-water evaluation in reservoir. Additionally, the original measurement data of array induction logging need to be processed by soft focusing, so as to obtain resistivity curves with radial detection depth of 10 inches to 120 inches and longitudinal resolution of 1 foot, 2 feet, and 4 feet, the original signal characteristics are overwhelmed by the conventional array induction logging through the soft focusing, as a result, the absolute value and difference characteristics of resistivity curves of array induction logging after synthesis are not very different when the reservoir porosity is low, so it is difficult to perform the oil-water evaluation in the reservoir accurately and effectively.

### SUMMARY

In view of the deficiencies of the prior art, the present invention particularly relates to an oil-water identification method based on conductivity dispersion characteristics of undisturbed formation. The present invention uses a novel method of identifying oil and water layers, the novel method is based on the dispersion of the original signal of array induction logging, mapping a spectrum image of array induction logging sub-array 7, and forming a two-dimensional oil-water identification chart by using the percent frequency effect (PFE) and the product of the resistivity after borehole correction and the formation porosity, and the test of actual logging data shows that this method can accurately and effectively divide the oil and water layers and improve the efficiency of the oil-water evaluation in reservoir.

A technical solution of the present invention is: an oil-water identification method based on resistivity dispersion characteristics of undisturbed formation, wherein:
step 1) acquiring a real part and imaginary part conductivity curves of the undisturbed formation of an array induction logging sub-array 7 and a formation porosity curve;
step 2) mapping an electrical spectrum curve of the array induction logging sub-array 7 based on the real part and imaginary part conductivity curve data of the array induction logging sub-array 7;
for the real part and imaginary part conductivity data of the array induction logging sub-array 7 with different frequencies obtained in step 1, mapping the real part and the imaginary part spectrum of the array induction logging sub-array 7 by using a logging mapping software;
step 3) calculating the PFE of the array induction logging sub-array 7 according to the real part conductivity data of the array induction logging sub-array 7 obtained in step 1);
for the real part conductivity data of the array induction logging sub-array 7 with different frequencies obtained in step 1), calculating and obtaining the percent frequency effect (PFE) based on the real part conductivity data (RPFE) according to the following formula:

$$RPFE = \frac{{σ}_{l} - {σ}_{h}}{{σ}_{l}}$$

where RPFE denotes the PFE calculated from the real part conductivity data, and σᵣ and σₕ are taken from the real part conductivity data of the array induction logging sub-array 7 with different frequencies, respectively;
step 4) constructing a two-dimensional oil-water identification chart according to the acquired RPFE data of the array induction logging sub-array 7 and a product of a resistivity after borehole correction of the array induction logging sub-array 7 and a formation porosity.

According to the oil-water identification method based on resistivity dispersion characteristics of undisturbed formation as described above, wherein in step 1), the array induction logging sub-array 7 is the one with a deepest detecting depth of the seven detecting sub-arrays of the array induction logging.

According to the oil-water identification method based on resistivity dispersion characteristics of undisturbed formation as described above, wherein in step 1), the real part and imaginary part conductivity refer to the real part and imaginary part conductivity curves at a plurality of different frequencies of the array induction logging sub-array 7 are selected based on 112 original conductivity curves measured by the array induction logging.

According to the oil-water identification method based on resistivity dispersion characteristics of undisturbed formation as described above, wherein in step 4), in the oil-water identification chart, the abscissa is the RPFE of the array induction logging sub-array 7, and the ordinate is the product of a synthesized resistivity of the array induction logging sub-array 7 original resistivity after borehole correction and the formation porosity.

According to the oil-water identification method based on resistivity dispersion characteristics of undisturbed formation as described above, wherein in step 4), the dispersion, and the product of the synthesized resistivity after borehole correction and the formation porosity is average values of a reservoir section.

The beneficial effect of the present invention is that the present invention constructs an oil-water evaluation method in reservoir based on the dispersion characteristics of real part conductivity, and improves the accuracy and interpretation efficiency of array induction logging in evaluating oil-water layers. The present invention uses the electrical dispersion effect of different conductivity curves of array induction logging sub-array 7 to divide the oil layer and water layer.

### BRIEF DESCRIPTION OF THE DRAWINGS

FIG. 1 is an evaluation result of a water layer of an array induction logging sub-array 7 spectrum.

FIG. 2 is an evaluation result of an oil layer of an array induction logging sub-array 7 spectrum.

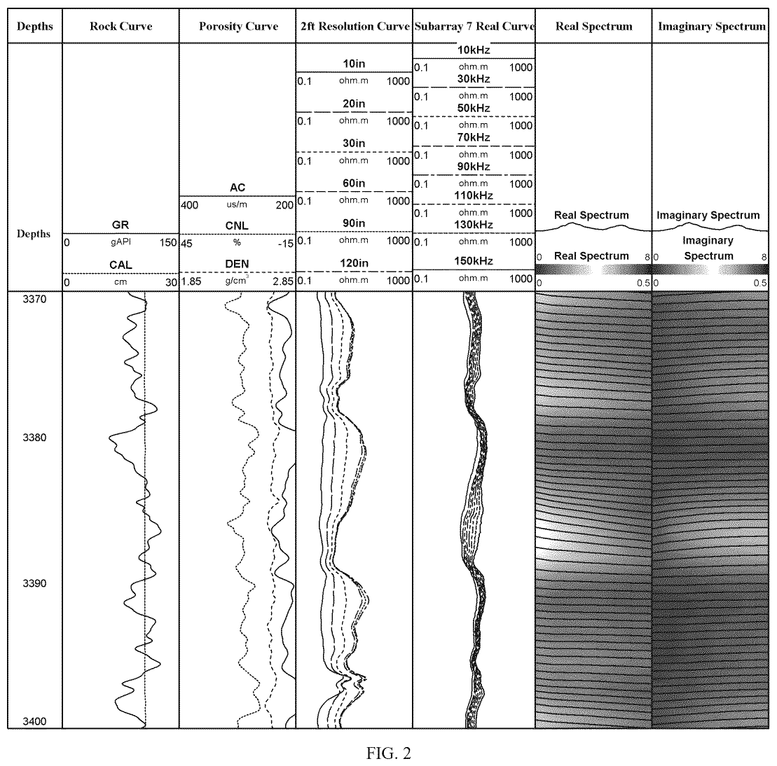

FIG. 3 is an array induction oil-water identification chart.

### DETAILED DESCRIPTION OF THE EMBODIMENTS

The technical scheme of the present invention is further explained below by drawings.

In order to achieve the above objective, the present invention provides a gas reservoir identification method based on the electrical dispersion effect of array induction logging, the specific implementation scheme is as follows:
step 1) the real part and the imaginary part conductivity curves of the undisturbed formation of the array induction logging sub-array 7 and the formation porosity curve are acquired;
the array induction logging sub-array 7 of the present invention is the one with the deepest detecting depth of the seven detecting sub-arrays of the array induction logging, that is, a long source distance sub-array.
step 2) the electrical spectrum curve of the array induction logging sub-array 7 is mapped based on the real part and imaginary part conductivity curve data of the array induction logging sub-array 7;
step 3) the conductivity dispersion of the array induction logging sub-array 7 is calculated according to the real part conductivity data of the obtained array induction logging sub-array 7;
step 4) the two-dimensional oil-water identification chart is constructed according to the acquired electrical dispersion data of the array induction logging sub-array 7 and the product of the resistivity after borehole correction of the array induction logging sub-array 7 and the formation porosity.

Further, in step 1), the real part and imaginary part conductivity refer to the real part and imaginary part conductivity curves at a plurality of different frequencies of the array induction logging sub-array 7 are selected based on 112 original conductivity curves measured by the array induction logging, such as real part conductivity curves for 8 frequencies of 10 kHz, 30 kHz, 50 kHz, 70 kHz, 90 KHz, 110 kHz, 130 kHz, and 150 kHz, and imaginary part conductivity curves for 8 frequencies of 20 kHz, 40 kHz, 60 kHz, 80 KHz, 100 kHz, 120 kHz, 140 kHz, and 160 kHz; the formation porosity curve refers to the formation porosity curve calculated by actual logging, the porosity is a known variable.

Further, in step 2), for the real part and imaginary part spectrum data of the array induction logging sub-array 7 with different frequencies obtained in step 1), the real part and the imaginary part spectrum of the array induction logging sub-array 7 are mapped by using the CIFlog logging mapping software, the oil-water layer can be preliminarily evaluated by using this spectrum. As in this embodiment, the real part spectrum and the imaginary part spectrum are the conductivity curves of eight consecutive frequencies of the array induction logging sub-array 7, and the calculation formula is the same as the dispersion used in the two-dimensional oil-water identification chart. The oil-water layer can be preliminarily identified independently by using this imaginary part spectrum and the real part spectrum.

Further, in step 3), for the real part conductivity data of the array induction logging sub-array 7 with different frequencies obtained in step 1, the PFE is calculated and obtained from the conductivity data according to the following formula:

$$RPFE = \frac{{σ}_{l} - {σ}_{h}}{{σ}_{l}}$$

where RPFE denotes the PFE calculated from the real part conductivity data, and σₗ and σₕ are taken from the real part conductivity data of the array induction logging sub-array 7 with the frequencies of 10 kHz and 150 kHz, respectively.

Further, in step 4), the oil-water identification chart constructed in step 4, wherein, the abscissa is the RPFE of the array induction logging sub-array 7, and the ordinate is the product of the synthesized resistivity of the array induction logging sub-array 7 original resistivity after borehole correction and the formation porosity. The PFE, the product of the synthesized resistivity after borehole correction and the formation porosity is the average values of the reservoir section. Then, according to the results of the oil testing and the relevant parameter values (RPFE and borehole corrected synthesized resistivity), the oil area and water area and oil-water area of the chart are divided, and the synthesized resistivity after borehole correction is a known variable.

### Analysis of Application Effect:

In order to better explain the present invention, the main content of the present invention will be further elucidated with reference to specific embodiments, but the content of the present invention is not limited to the following embodiments.

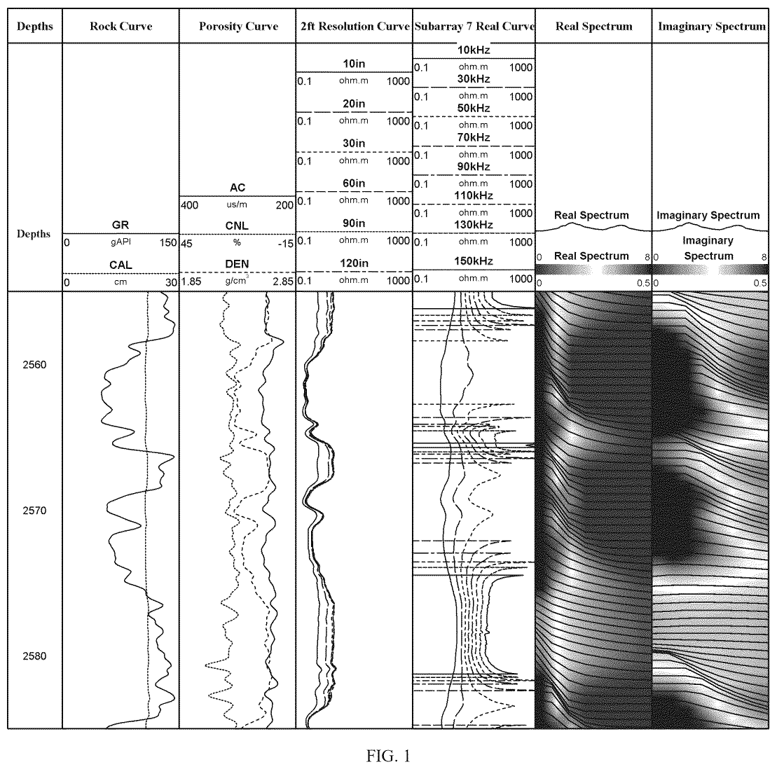

In the method, firstly, the original conductivity logging data of the array induction sub-array 7 is obtained, and then the real part and imaginary part electrical spectrum curve of the array induction logging sub-array 7 are constructed according to the conductivity data (as shown in FIG. 1), in the upper part of the well section (2555 m to 2575 m), the spectrum value of the array induction logging sub-array 7 decreases from a large value to a small value, (in the logging interpretation software, lines and two colors can be used to denote the high value and low value of the spectrum respectively, and oil-water evaluation of the reservoir is performed according to the color change, so as to improve the interpretation efficiency), and it is judged that two adjacent layers of the water layer are consistent with the resistivity logging curve and the gamma logging curve; In the lower part of the well section (3370 m to 3400 m), the spectrum value of array induction logging sub-array 7 is relatively small, and according to the method of the present invention, three adjacent oil layers are judged, which is consistent with the conclusion of the oil testing. This method can be used to evaluate the reservoir with high accuracy and identification, which can greatly improve the efficiency of logging interpretation.

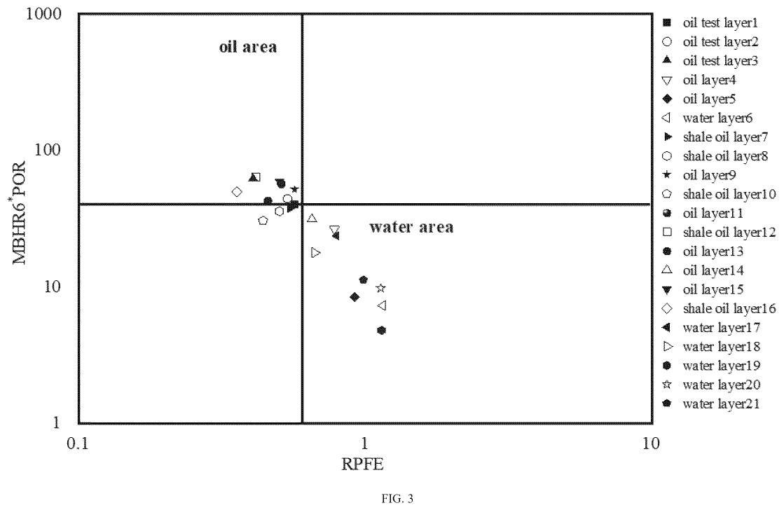

Based on the PFE of the array induction logging sub-array 7 and the corrected resistivity of the original borehole, the two-dimensional oil-water identification chart (as shown in FIG. 3) is mapped, the abscissa of the chart is the PFE and the ordinate of the chart is the original resistivity of array induction logging sub-array 7 after borehole correction, the oil area, water area and oil-water area can be divided on the chart by combining with the oil testing conclusion and the logging curve; When this chart is used to identify other reservoirs in the current area, accurate oil-water evaluation of the target reservoir can be performed only by obtaining the PFE of array induction logging sub-array 7 and the original resistivity of array induction logging sub-array 7 after borehole correction. This method is compared to single array induction synthesis resistivity for oil-water evaluation of reservoir, and its accuracy is greatly improved.

What is claimed is:

1. An oil-water identification method based on RPFE characteristics of an undisturbed formation, comprising the following steps:
step 1): acquiring a real part conductivity curve and an imaginary part conductivity curve of the undisturbed formation of an array induction logging sub-array 7 and a formation porosity curve;
step 2): mapping an electrical spectrum curve of the array induction logging sub-array 7 based on real part and imaginary part conductivity curve data of the array induction logging sub-array 7;
for real part and imaginary part conductivity data of the array induction logging sub-array 7 with different frequencies obtained in the step 1), mapping a real part and an imaginary part spectrum of the array induction logging sub-array 7 by using a logging mapping software;
step 3): calculating the PFE of the array induction logging sub-array 7 according to the real part conductivity curve data of the array induction logging sub-array 7;
for the real part conductivity curve data of the array induction logging sub-array 7 with the different frequencies obtained in the step 1), calculating and obtaining the RPFE according to the following formula:
$$RPFE = \frac{{σ}_{l} - {σ}_{h}}{{σ}_{l}}$$
wherein RPFE denotes the PFE calculated from the real part conductivity data, and σₗ and σₕ are taken from the real part conductivity curve data of the array induction logging sub-array 7 with 10 kHz and 150 kHz, respectively;
step 4): constructing a two-dimensional oil-water identification chart according to the RPFE of the array induction logging sub-array 7 and a product of a resistivity after borehole correction of the array induction logging sub-array 7 and a formation porosity.

2. The oil-water identification method based on the RPFE characteristics of the undisturbed formation according to claim 1, wherein in the step 1), the array induction logging sub-array 7 has a deepest detecting depth of seven array induction logging sub-arrays.

3. The oil-water identification method based on the RPFE characteristics of the undisturbed formation according to claim 1, wherein in the step 1), a real part and an imaginary part conductivity refer to the real part and the imaginary part conductivity curves at a plurality of different frequencies of a reaction undisturbed formation of the array induction logging sub-array 7, wherein the real part and the imaginary part conductivity curves are selected based on 112 original conductivity curves measured by array induction logging.

4. The oil-water identification method based on the RPFE characteristics of the undisturbed formation according to claim 1, wherein in the step 4), in the two-dimensional oil-water identification chart, the abscissa is the conductivity dispersion of the seventh array induction logging sub-array, and the ordinate is the product of the resistivity after the borehole correction of the array induction logging sub-array 7 and the formation porosity.

5. The oil-water identification method based on the RPFE characteristics of the undisturbed formation according to claim 4, wherein in the step 4), the RPFE and the product of the resistivity after the borehole correction and the formation porosity are average values of a reservoir section.
